(19) United States
(12) Patent Application Publication
(10) Pub. No.: US 2025/0302290 A1
(43) Pub. Date: Oct. 2, 2025

(54) LARYNGOSCOPE PARTICLE EVACUATOR AND USES THEREOF

(71) Applicants: Anand Devaiah, Brookline, MA (US); Ruhaan Bhagat, Chicago, IL (US); Zixin Guan, Patterson, CA (US); Zixian He, Patterson, CA (US); Camila Zhan Jin, Brooklyn, NY (US); Tommaso Ranzani, Cambridge, MA (US); Yousuf Ihsan Shehadi, New York, NY (US)

(72) Inventors: Anand Devaiah, Brookline, MA (US); Ruhaan Bhagat, Chicago, IL (US); Zixin Guan, Patterson, CA (US); Zixian He, Patterson, CA (US); Camila Zhan Jin, Brooklyn, NY (US); Tommaso Ranzani, Cambridge, MA (US); Yousuf Ihsan Shehadi, New York, NY (US)

(21) Appl. No.: 19/088,240

(22) Filed: Mar. 24, 2025

Related U.S. Application Data
(60) Provisional application No. 63/570,562, filed on Mar. 27, 2024.

(51) Int. Cl. A61B 1/267; A61B 1/00; A61B 1/12

(52) U.S. Cl. CPC A61B 1/267; A61B 1/00128; A61B 1/126

(57) ABSTRACT

An evacuator device for attachment to a laryngoscope has a rectangular body with a proximal end, a distal end, and a curved region between the proximal end and the distal end. The body defines an interior passage extending from the proximal end to the distal end. The proximal end is configured to attach to a suction tube of a vacuum source. The distal end is configured to attach to a blade of the laryngoscope. A diameter of the interior passage is sized to allow airborne particles and fluid from a patient's larynx and pharynx to be evacuated through the interior passage due to the negative pressure created by the vacuum source. The curved region reduces obstruction of a user's view of the patient's larynx and pharynx.

### CROSS-REFERENCE TO RELATED APPLICATIONS

This application claims priority to and benefit of U.S. Provisional Application No. 63/570,562, filed Mar. 27, 2024, entitled LARYNGOSCOPE PARTICLE EVACUATOR AND USES THEREOF, the contents of which are incorporated herein by reference in their entirety for all purposes.

### BACKGROUND

A laryngoscope is a medical instrument that aids a doctor or other healthcare professional in the visualization of a patient's larynx and pharynx. Laryngoscopy can be used, for example, in endotracheal intubation, the administration of anesthetic and respiratory gasses, other medical procedures. In direct laryngoscopy, a user inserts the laryngoscope into the patient's mouth and then manipulates the instrument to allow the user a direct line of sight of the patient's larynx and pharynx. However, during these procedures, the patient's breathing airway is opened, thereby placing the user at a much higher risk for contracting airborne diseases from the patient. Moreover, improving the safety of laryngoscopy is difficult because procedures such as intubation are complex medical procedures that require the user to be in close contact with the patient.

Some current tools that aim to increase the safety of laryngoscopy include video laryngoscopes that use fiber optic or digital cameras for indirection visualization of the larynx and pharynx. However, these tools do not completely eliminate the risk of contagion and are much more expensive than traditional laryngoscopes. Another common technique is to place a plastic sheet barrier over the patient's head, creating a physical separation between the user performing the intubation and the patient's breathing airway. However, while this technique does reduce the pathogen exposure during the procedure, it also limits access to the larynx and pharynx, making it much more challenging for the user to perform the intubation. Furthermore, the large size of the plastic sheet makes the process of clean up and disposal difficult and time-consuming, adding to the workload of the procedure and increasing the chance of cross-contamination.

### SUMMARY

This disclosure describes a laryngoscope particle evacuator that can attach to common suction sources available in operating rooms, hospitals, clinics, and emergent care settings. The vacuum source creates a negative pressure zone within the particle evacuator to capture the patient's pathogen load during laryngoscopy. In some embodiments, the particle evacuator is a device that attaches to a standard direct laryngoscope. In other embodiments, the particle evacuator is directly incorporated into the laryngoscope. Advantageously, the particle evacuator can significantly reduce the number of aerosolized pathogens that escape into the operating room during laryngoscopy, potentially preventing the spread of infections not only in hospitals and clinics but also in other settings where similar procedures are performed.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

Reference to the detailed description, combined with the following figures, will make the disclosure more fully understood, wherein:

FIG. 1 illustrates a prior art laryngoscope for use with an endotracheal intubation;

FIGS. 2A and 2B illustrate an evacuator device, according to an embodiment of the disclosure;

FIG. 2C illustrates the evacuator device attached to a laryngoscope, according to an embodiment of the disclosure;

FIGS. 3A and 3B illustrate alternative designs of the evacuator device, according to embodiments of the disclosure;

FIG. 4 illustrates the use of the evacuator device during an endotracheal intubation, according to an embodiment of the disclosure; and

FIGS. 5A and 5B illustrate an integrated laryngoscope and evacuator assembly, according to an embodiment of the disclosure.

### DETAILED DESCRIPTION

In the following description, like components have the same reference numerals, regardless of different illustrated examples. To illustrate examples clearly and concisely, the drawings may not necessarily reflect appropriate scale and may have certain features shown in somewhat schematic form. The disclosure may describe and/or illustrate features in one example, and in the same way or in a similar way in one or more other examples, and/or combined with or instead of the features of the other examples.

In the specification and claims, for the purposes of describing and defining the invention, the terms “about” and “substantially” represent the inherent degree of uncertainty attributed to any quantitative comparison, value, measurement, or other representation. The terms “about” and “substantially” moreover represent the degree by which a quantitative representation may vary from a stated reference without resulting in a change in the basic function of the subject matter at issue. Open-ended terms, such as “comprise,” “include,” and/or plural forms of each, include the listed parts and can include additional parts not listed, while terms such as “and/or” include one or more of the listed parts and combinations of the listed parts. Use of the terms “upper,” “lower,” “above,” “below” and the like helps only in the clear description of the disclosure and does not limit the structure, positioning and/or operation of the disclosure in any manner.

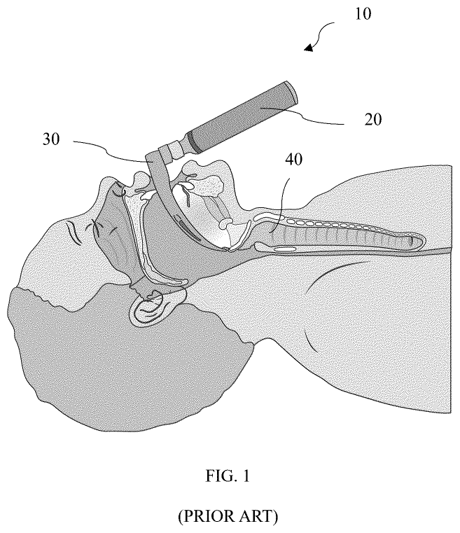

FIG. 1 illustrates a prior art laryngoscope 10 that may be used, for example, during an endotracheal intubation. As shown in FIG. 1, the laryngoscope 10 may generally include a handle 20 and a blade 30 extending from the handle 20. The handle 20 may be configured to extend out of the patient's mouth and be manipulated by the user. The blade 30 may be configured to be inserted into the patient's breathing airway and be used to lift the tongue and/or epiglottis to allow the user a direct view of the larynx and pharynx. Some current laryngoscopes 10 also include a means to allow for insertion of an endotracheal tube into the patient's trachea 40.

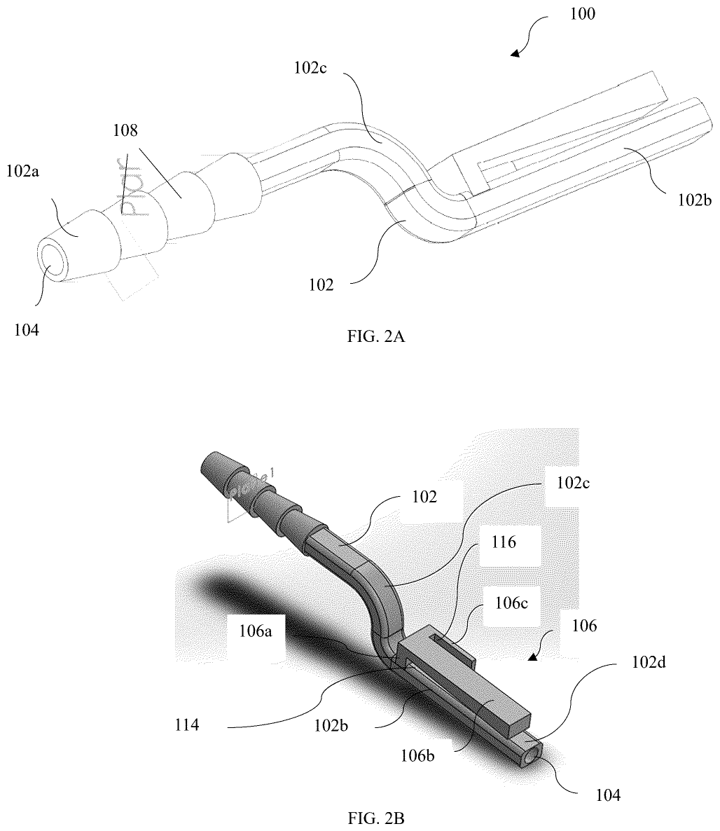

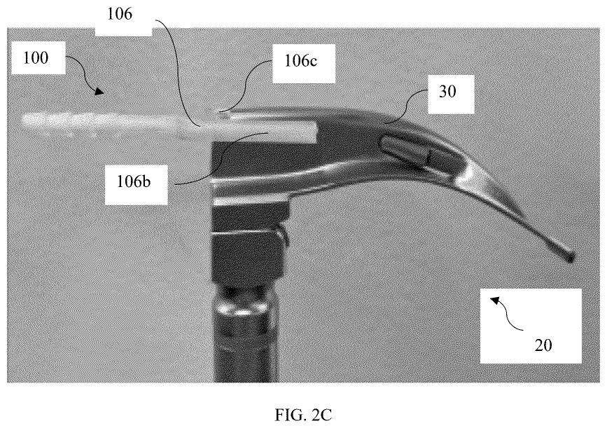

FIGS. 2A and 2B illustrates an evacuator device 100 for attachment to a laryngoscope, according to an embodiment of the disclosure. As shown in FIG. 2A, the evacuator device 100 may comprise a substantially rectangular body 102 having a proximal end 102a and a distal end 102b. In embodiments, the corners of the body 102 may be rounded to reduce the overall size of the body 102 and for more comfortable handling by the user. A curved region 102c may be formed in the body 102 between the proximal end 102a and the distal end 102b such that the body 102 forms an “S” shape. The body 102 may define an interior passage 104 extending between the proximal end 102a and the distal end 102b. The proximal end 102a may be configured to attach to a suction tube of a vacuum source (not shown). For example, the proximal end 102a may comprise a plurality of cone-shaped barbs 108 disposed about the proximal end 102a for forming an interference fit with an interior of the suction tube. However, the disclosure contemplates that the proximal end 102a could include other suitable means of attaching to a suction tube such as a luer lock, adjustable pressure valve, stop valve, or other connection. A diameter of the interior passage 104 may be sized to allow airborne particles and fluid from the larynx and pharynx to be evacuated through the interior passage 104 due to the negative pressure created by the vacuum source. An angle of the curved region 102c may be selected to reduce obstruction of the user's field of vision while maintaining sufficient suction capacities through the interior passage 104. The body 102 may comprise an inexpensive and readily available plastic material such as polylactic acid (PLA). However, the disclosure contemplates that the body 102 could comprise other suitable plastics, such as polyethylene (PE), polyurethane, polytetrafluoro-ethylene (PTFE), polyethersulfone (PES), polyethylene-terepthalate, or polyetherether ketone (PEEK). The body 102 could also comprise medical grade metals such as stainless steel, titanium, copper, or aluminum.

As shown in FIG. 2B, the distal end 102b of the body 102 may include a clip 106 formed integrally with the body 102. In embodiments, the clip 106 may comprise a first section 106a positioned adjacent the curved region 102c and extending upward from the distal end 102b of the body 102. A second section 106b may extend distally from the first section 106a and may slope downward toward a top surface 102d of the distal end 102b such that a tapered first slot 114 is formed between the second section 106b and the top surface 102d. A third section 106c may also extend distally from the first section 106a. The third section 106c may be spaced apart from the second section 106b such that a second slot 116 is formed between the third section 106c and the second section 106b. The third section 106c may have a length that is selected to be less than a length of the second section 106b. However, the disclosure contemplates that the third section 106c could have a length that is equal to or longer than a length of the second section 106b.

FIG. 2C illustrates the evacuator device 100 attached to a blade 30 of a laryngoscope 10, according to an embodiment of the disclosure. As shown in FIG. 2C, a user may removeably attach the evacuator device 100 to the blade 30 such that the second section 106b of the clip 106 extends into an interior of the blade 30 and the third section 106c extends along a top surface of the blade 30. As such, the clip 106 may form a secure attachment to the blade 30 at two different attachment points—i.e., at both the first tapered slot 114 and the second slot 116. Moreover, the rectangular cross-section of the body 102 provides a surface area for contacting the blade 30 sufficient to increase the stability of the attachment between the evacuator device 100 and the blade 30. However, the disclosure also contemplates other suitable methods of attaching the evacuator device 100 to the blade 30 such as a hinge, spring clips, set screws, pins, or spring pins.

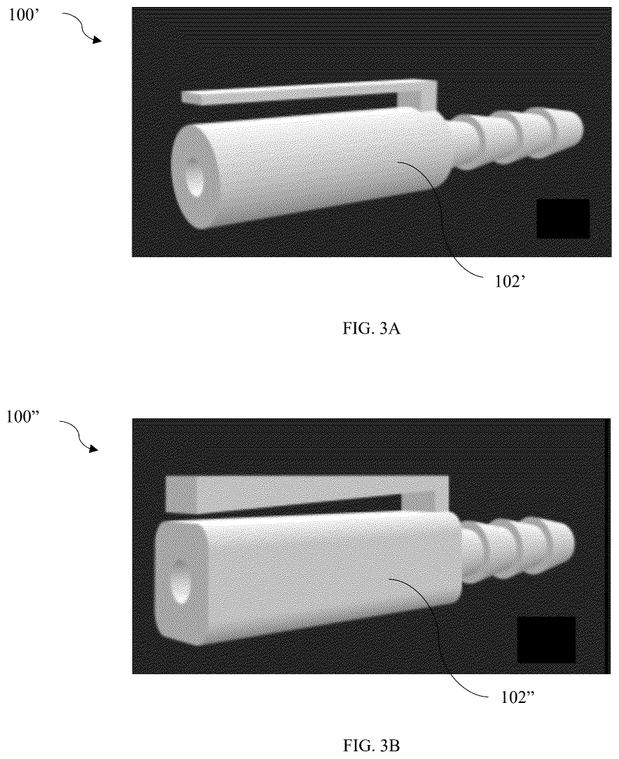

FIGS. 3A and 3B illustrate alternative designs of the evacuator device 100′, 100″ according to embodiments of the disclosure. As shown in FIG. 3A, the evacuator device 100′ is substantially the same as the evacuator device 100 except that the body 102′ has a round cross section and lacks a curved region in the body 102′. As shown in FIG. 3B, the evacuator device 100″ is substantially the same as the evacuator device 100 except that the body 102″ has a rectangular cross section and lacks a curved region in the body 102″.

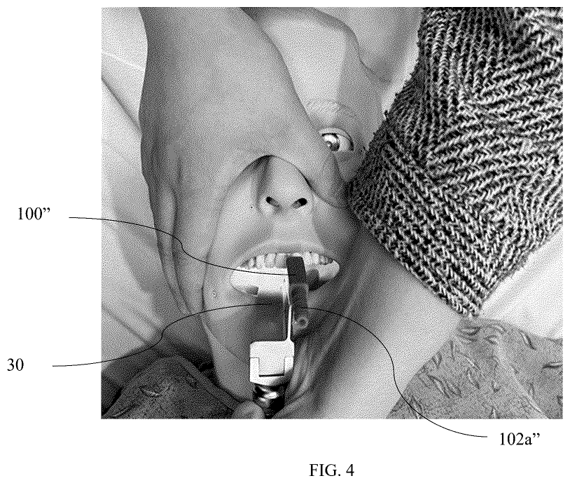

FIG. 4 illustrates the use of the evacuator device 100″ during an endotracheal intubation, according to an embodiment of the disclosure. While the use of the evacuator device 100″ is specifically illustrated, it will be appreciated that any of the evacuator devices 100, 100′ or 100″ could be used during the intubation. As shown in FIG. 4, before inserting the blade 30 into the patient's breathing airway, a user may securely attach the evacuator device 100″ to the blade 30. Once the blade 30 is properly inserted into the airway, the user may attach a suction tube of a vacuum source to the proximal end 102a″ of the evacuator device 100″. The user may then actuate the vacuum source for a period of time or leave suction on continuously through the procedure effective to remove airborne particles and fluid from the patient's larynx and pharynx. In embodiments, the period of time may be about 30 seconds. However, the disclosure contemplates that the period of time may be longer or shorter than 30 seconds.

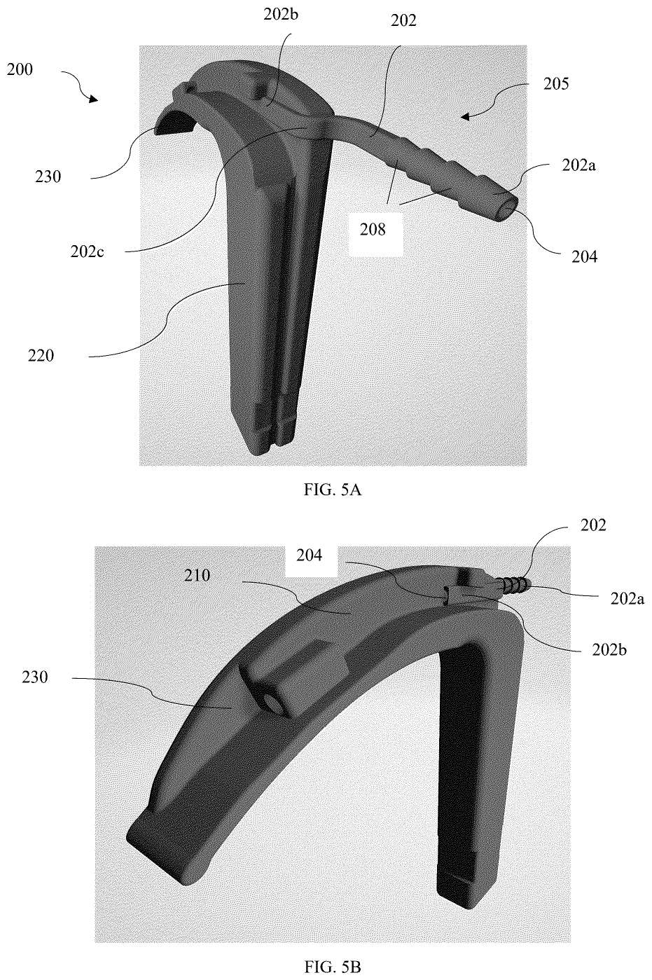

FIGS. 5A and 5B illustrate a laryngoscope and evacuator assembly 200, according to an embodiment of the disclosure. As shown in FIG. 5A, the assembly 200 may comprise a handle 220 and a blade 230 extending from the handle 220. An evacuator portion 205 of the assembly may include a substantially rectangular body 202 having a proximal end 202a and a distal end 202b. In embodiments, the corners of the body 202 may be rounded to reduce the overall size of the body 202. A curved region 202c may be formed in the body 202 between the proximal end 202a and the distal end 202b such that the body 202 forms an “S” shape that curves around an edge of the blade 30. The body 202 may define an interior passage 204 extending between the proximal end 202a and the distal end 202b. The proximal end 202a may be configured to attach to a suction tube of a vacuum source. For example, the proximal end 202a may comprise a plurality of cone-shaped barbs 208 disposed about the proximal end 202a for forming an interference fit with the interior of the suction tube. A diameter of the interior passage 204 may be sized to allow airborne particles and fluid to be evacuated from the larynx and pharynx through the interior passage 204 due to the negative pressure created by the vacuum source. An angle of the curved region 202c may be selected to reduce obstruction of the user's field of vision while maintaining sufficient suction capacities through the interior passage 204. As shown in FIG. 5B, the distal end 202b of the body 202 may be formed integrally with the blade 230 such that a side surface 210 of the blade 230 at least partially defines the interior passage 204. During an endotracheal intubation, once the blade 230 is properly inserted into the patient's breathing airway, the user may attach a suction tube of a vacuum source to the proximal end 202a of the evacuator portion 205 and actuate the vacuum source for a period of time effective to remove airborne particles and fluid from the larynx and pharynx.

While the disclosure particularly shows and describes preferred examples, those skilled in the art will understand that various changes in form and details may exist without departing from the spirit and scope of the present application as defined by the appended claims. The scope of this present application intends to cover such variations. As such, the foregoing description of examples of the present application does not intend to limit the full scope conveyed by the appended claims.

We claim:

1. An evacuator device for attachment to a laryngoscope, the evacuator device comprising:
a substantially rectangular body having a proximal end, a distal end, and a curved region formed in the body between the proximal end and the distal end, the body defining an interior passage extending from the proximal end to the distal end, the proximal end configured to attach to a vacuum source, and the distal end configured to attach to a blade of the laryngoscope;
wherein a diameter of the interior passage is sized to allow airborne particles and fluid from a patient's larynx and pharynx to be evacuated through the interior passage due to a negative pressure created by the vacuum source; and
wherein the curved region reduces obstruction of a user's view of the patient's larynx and pharynx.

2. The evacuator device of claim 1, wherein the body comprises a plastic or metal material.

3. The evacuator device of claim 2, wherein the plastic material is polylactic acid (PLA).

4. The evacuator device of claim 1, wherein the proximal end of the body comprises a plurality of cone-shaped barbs for forming an interference fit with an interior diameter of a suction tube of the vacuum source.

5. The evacuator device of claim 1, wherein the distal end of the body comprises a clip for securing the evacuator device to the blade.

6. The evacuator device of claim 5, wherein the clip is formed integrally with the body.

7. The evacuator device of claim 5, wherein the clip comprises a first section positioned adjacent the curved region and extending upward from the distal end of the body, and a second section extending distally from the first section.

8. The evacuator device of claim 7, wherein the second section slopes downward toward a top surface of the distal end such that a tapered first slot is formed between the second section and the top surface.

9. The evacuator device of claim 7, wherein the second section is configured to extend into an interior of the blade when the evacuator device is attached to the blade.

10. The evacuator device of claim 7, wherein the clip comprises a third section extending distally from the first section, the third section spaced apart from the second section such that a second slot is formed between the third section and the second section.

11. The evacuator device of claim 10, wherein a length of the third section is selected to be less than a length of the second section.

12. The evacuator device of claim 10, wherein the third section is configured to extend along a top surface of the blade when the evacuator device is attached to the blade.

13. A method of evacuating airborne particles and fluid from a larynx and pharynx of a patient during a medical procedure, the method comprising:
attaching an evacuator device to a blade of a laryngoscope, evacuator device comprising a substantially rectangular body having a proximal end, a distal end, and a curved region formed in the body between the proximal end and the distal end, the body defining an interior passage extending from the proximal end to the distal end, the proximal end configured to attach to a vacuum source, and the distal end configured to attach to the blade of the laryngoscope;
inserting the blade into a breathing airway of the patient; and
attaching the vacuum source to the proximal end of the body;
wherein a diameter of the interior passage is sized to allow the airborne particles and fluid from the patient's larynx and pharynx to be evacuated through the interior passage due to a negative pressure created by the vacuum source; and
wherein the curved region reduces obstruction of a user's view of the patient's larynx and pharynx.

14. The method of claim 13, wherein the medical procedure is an endotracheal intubation.

15. The method of claim 13, wherein attaching the evacuator device to the blade comprises attaching a clip on the distal end of the body to the blade.

16. The method of claim 15, wherein attaching the clip on the distal end of the body to the blade comprises attaching the clip at two different attachment points to the blade.

17. A laryngoscope and evacuator assembly comprising:
a handle;
a blade extending from the handle; and
an evacuator portion, the evacuator portion comprising a substantially rectangular body having a proximal end, a distal end, and a curved region formed in the body between the proximal end and the distal end, the body defining an interior passage extending from the proximal end to the distal end, the proximal end configured to attach to a vacuum source;
wherein a diameter of the interior passage is sized to allow airborne particles and fluid from a patient's larynx and pharynx to be evacuated through the interior passage due to a negative pressure created by the vacuum source; and
wherein the curved region reduces obstruction of a user's view of the patient's larynx and pharynx.

18. The laryngoscope and evacuator assembly of claim 17, wherein the curved region extends around an edge of the blade.

19. The laryngoscope and evacuator assembly of claim 17, wherein the proximal end of the body comprises a plurality of cone-shaped barbs for forming an interference fit with an interior diameter of a suction tube of the vacuum source.

20. The laryngoscope and evacuator assembly of claim 17, wherein the distal end of the body is formed integrally with the blade such that a side surface of the blade at least partially defines the interior passage.
